(19) United States
(12) Patent Application Publication
(10) Pub. No.: US 2018/0110472 A1
(43) Pub. Date: Apr. 26, 2018

(54) METHOD AND SYSTEM FOR DETERMINING STATUS OF PREDIABETES IN AN INDIVIDUAL

(71) Applicant: Quattro Folia Oy, Espoo (FI)

(72) Inventors: Pekka Lönnroth, Espoo (FI); Petteri Väisänen, Tampere (FI); Harri Okkonen, Espoo (FI); Ari Sinisalo, Helsinki (FI); Markku Saraheimo, Helsinki (FI); Niina Nippula, Veikkola (FI)

(73) Assignee: Quattro Folia Oy, Espoo (FI)

(21) Appl. No.: 15/331,261

(22) Filed: Oct. 21, 2016

(51) Int. Cl. A61B 5/00; A61B 5/145; A61B 10/00; G06F 19/00

(52) U.S. Cl. CPC A61B 5/7275; A61B 5/14532; A61B 10/0051; G06F 19/322; G06F 19/345; G06F 19/3431; G06F 19/3418; A61B 5/7282

(57) ABSTRACT

A system determines onset of a prediabetic state and associated risk of type 2 diabetes without evidential symptoms. In an implementation, a computer implemented method for monitoring an onset and progress of prediabetes in an individual includes periodically capturing an insulin level of an individual and a blood glucose level of the individual over a predetermined time interval for a predefined period, deriving an insulin production trend or a relative insulin resistivity trend over the predefined period, wherein the relative insulin resistivity is a ratio of an insulin level and a blood glucose level, wherein the trends are indicative of trend categories comprising an increasing trend, a steady trend and a decreasing trend, and determining a status of prediabetes in the individual based on at least one of the insulin production trend, relative insulin resistivity trend, and personal data of the individual.

### FIELD OF INVENTION

The present invention generally relates to the field of diabetes and prediabetes management. More specifically, the present invention relates to a system and method for determining the status, onset and progress of prediabetes in an individual to prevent further prognosis of T2D development in an individual.

### BACKGROUND OF INVENTION

Diabetes, especially Type 2 diabetes (T2D) is increasing exponentially all over the world and T2D unlike type 1 diabetes (T1D) develops evident symptoms detrimental for appropriate diagnosis only over a gradual period of time thereby making early diagnosis a difficult ordeal. Traditionally diabetes is diagnosed using various laboratory tests. Due to the nature of type 2 diabetes, (T2D) development, laboratory tests may give false ‘healthy’ results although T2D is developing, e.g., fasting blood glucose value is in normal range due to increased insulin production. The insulin level is, however, not usually measured and therefore development of T2D is not diagnosed.

Furthermore, another condition prevalent in individuals whereby the onset of diabetes has not occurred, but will go on to develop diabetes eventually in future years is termed as prediabetes, which in other words is impaired glucose tolerance. In the prediabetes phase, the body tissues' ability to utilize insulin becomes lower which is compensated by increased insulin production from pancreas and, thus, blood glucose levels do not increase until the pancreas lose the ability to satisfy the increased insulin demand. The prediabetes phase, also called increased insulin resistivity of the body therefore signifies increased insulin resistivity already present with healthy levels of blood glucose measurements although the process that leads to T2D has already started. Also, normal blood glucose fasting measurements are not indicative of the prediabetic phase of an individual, thereby requiring better diagnosis and management of prediabetes, and diabetes.

In light of the above, there is a need for a system that determines the onset of a prediabetic state and the associated risk of T2D without evidential symptoms suggesting the same. A system suggesting appropriate life style changes to slow down or stop the start of the prediabetic phase is also needed.

### BRIEF DESCRIPTION OF DRAWINGS

The accompanying figures wherein like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is illustrative of a system 100 for monitoring an onset and progress of prediabetes in an individual in accordance with an embodiment of the present application.

FIG. 2 is illustrative of a flow diagram of a computer implemented method monitoring the onset and progress of the prediabetes in an individual.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present application.

### DETAILED DESCRIPTION OF THE INVENTION

Before describing in detail embodiments that are in accordance with the present invention, it should be observed that the embodiments reside primarily in a method and system for monitoring an onset and progress of prediabetes in an individual. Accordingly, the method steps and system components have been represented where appropriate by conventional symbols in the drawings, showing only those specific details that are pertinent to understanding the embodiments of the present application so as not to obscure the disclosure with details that will be readily apparent to those of ordinary skill in the art having the benefit of the description herein.

In this document, the terms “comprises,” “comprising,” or any other variation thereof, are intended to cover a non-exclusive inclusion, such that a process, method, article, or apparatus that comprises a list of objects may include not only those objects but also include other objects not expressly listed or inherent to such process, method, article, or apparatus. An object proceeded by “comprises . . . a” does not, without more constraints, preclude the existence of additional identical objects in the process, method, article, or apparatus that comprises the object.

Embodiments of the present invention provide a method and system for monitoring the status, onset and progress of prediabetes and associated risks of onset of type 2 diabetes of an individual.

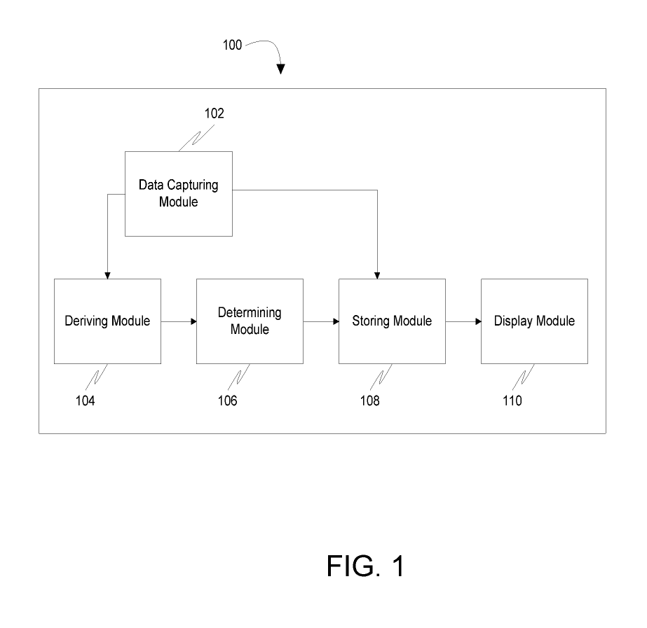

FIG. 1 illustrates a system 100 for monitoring an onset and progress of prediabetes in an individual in accordance with an embodiment of the present invention. As illustrated in FIG. 1, system 100 includes one or more data capturing modules 102 configured to capture insulin and glucose related data of an individual at predetermined time intervals over a predefined period. The predetermined time intervals over a predefined period may be coordinated based on an initial (baseline) set of measurements as determined in the individual. In some examples, an individual's insulin and glucose related data as a fasting requirement is captured and measured for example for five days continuously to establish baseline set of measurements, followed by measurements at predetermined time intervals over a time period of for example two weeks based on the initial (baseline) measurements. In some embodiments, the predefined period may include one or more time periods sufficient to provide trend information or sufficient to provide analysis of the trends in response to suggestions and changes prescribed to the individual.

Data capturing module 102 can be any device configured to capture insulin and glucose related data. For instance, data capturing module 102 can be one of, but not limited to, a mobile phone, a smartphone, a portable device, a tablet device, a wearable computer, a smart swatch, a laptop and a desktop computer, an add-on device or a combination thereof, configured to periodically capture data. In accordance with the invention, the insulin levels of the individual are primarily measured by non-invasive means but in some cases they may be from invasive origin. The means of measuring insulin can be extracted from one of, but not limited to blood sample, a saliva sample, a tears sample and a sweat sample. In some examples, insulin levels can be estimated from C-peptide levels; means of measuring C-peptide levels can be extracted from one of, but not limited to capillary blood sample, plasma blood sample, a saliva sample, a tears sample and a sweat sample. Insulin estimation to blood level insulin from plasma, saliva, tear or sweat measurements may be calculated using a mathematical model, the mathematical model may include but is not limited to Monte Carlo Tree Search, Neural Network Optimization or any other Artificial Intelligence. The Mathematical model may include one or more personally adaptive factors that may change over time. In some examples, insulin may also be estimated using an advanced continuous glucose monitoring (aCGM) system.

The glucose related data includes blood glucose levels measured from capillary blood samples. In another embodiment, blood glucose levels may be estimated using plasma glucose samples or samples of saliva, tears or sweat, the means of measuring glucose can be one of, but not limited to finger stick, saliva, tears and sweat. Blood glucose estimation from plasma, saliva, tears or sweat glucose measurements may be calculated using a mathematical model, the mathematical model may include but is not limited to Monte Carlo Tree Search, Neural Network Optimization or any other Artificial Intelligence. In some examples, blood glucose may also be estimated using a continuous glucose monitoring (CGM) system. The Mathematical model may include one or more personally adaptive factors that may change over time.

Data capturing module 102 may also be configured to capture personal data of the individual. Personal data of the individual is captured contextually, wherein personal data may include, but is not limited to body mass index (BMI), waist circumflex (WC), sex, age, physical activity details, eating habit details, lifestyle details, genome related information and any other existing diseases' details. The personal data may be consumed by the mathematical model in the prediction calculation.

Data capturing module 102 may include one or more sensors attached to one or more portions of the body of the individual, or may be a sensor itself. The one or more sensors may include one or more of, but not limited to, an energy source MCU, Bluetooth radio BLE, 24-bit sigma delta ADC, 32- or more bit processor ARM, memory, rechargeable battery, bracket for test assay, LED light sources in one or more arrays of wavelengths white (400 . . . 700 nm)/red (700 nm)/green (570 nm)/NIR (near infrared 1550 nm) and photodiode detectors capable to sense emitted wavelengths. Module may be equipped or attached temporarily with elements capable to spectroscopy in radio frequencies.

System 100 as per FIG. 1 further includes a deriving module 104, configured to derive at least one of an insulin production trend and a relative insulin resistivity trend. The relative insulin resistivity is a ratio between the actual or calculated insulin and glucose levels in blood. The insulin production trend and relative insulin resistivity trends are indicative of trend categories comprising an increasing trend, a steady trend and a decreasing trend. In an embodiment in accordance with the invention the relative insulin resistivity is a ratio between the blood insulin level and blood glucose level.

Referring back to FIG. 1, system 100 further includes a determining module 106, wherein determining module 106 is configured to determine a status of prediabetes in the individual based on at least one of the relative insulin resistivity trend, insulin production trend for the individual, BMI, WC and other personal data of the individual. The personal data as captured by data capturing module 102 may be detrimental in further defining the trends of the insulin level and insulin resistivity trend.

As illustrated in FIG. 1, system 100 also includes a storing module 108 for storing the insulin level and the blood glucose level of the individual, the insulin level and blood glucose level as captured by the system 100 in data capturing module 102. The stored values in storing module 108 may be scaled to values as measured from capillary blood sample. Storing module 108 may be located in a computing device of the individual, wherein a computing device is at least one of a smartphone, a tablet, a laptop, a desktop, a wearable computer, a smartwatch, or a combination thereof. In another embodiment in accordance with the present invention, storing module 108 is hosted on a cloud based server. Numerous individuals participating in system 100 can be accessed through the cloud based server, wherein each individual can have a personal account accessed through a device like a smartphone.

System 100 also includes a display module 110 which is configured to display the trends and status of prediabetes. Display module 110 displays trend categories of the insulin production trend and relative insulin resistivity trend, wherein trend is indicative of trend categories comprising an increasing trend, a steady trend and a decreasing trend. Display module 110 may further present suggestions for changes in lifestyle or eating habits, or motivating hints, or both based on the progress of the individual.

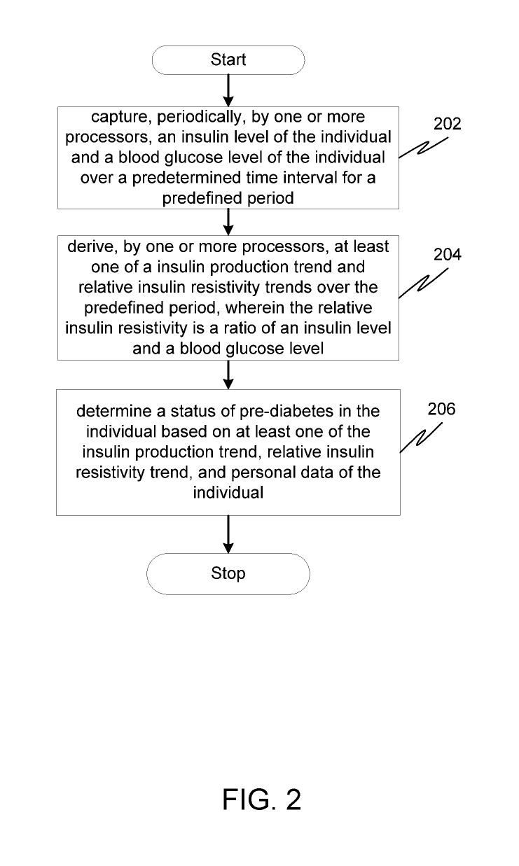

FIG. 2 illustrates a flow diagram of a computer implemented method for monitoring the onset and progress of prediabetes in an individual. The method in accordance of the method can be implemented by system 100 or parts thereof.

To begin the process, at step 202, data capturing module 102 captures insulin production data, glucose related data and personal data periodically as per the context of the individual, wherein insulin production level and blood glucose levels of the individual in lieu of the personal data of the individual are captured and measured. The insulin production data, glucose related data and personal data are captured at predetermined time intervals over predefined period in accordance with system 100. In accordance with the invention, the insulin levels of the individual are primarily measured by non-invasive means but may also be from invasive origin. The means of measuring insulin can be extracted from one of, but not limited to blood sample, a saliva sample, a tears sample and a sweat sample. In some examples, insulin levels can be estimated from C-peptide levels, means of measuring C-peptide levels that can be extracted from one of, but not limited to blood sample, a saliva sample, a tears sample and a sweat sample. Actual or calculated levels of blood glucose and insulin levels, will get adjusted mathematically in line with capillary blood sample measurements.

The glucose related data at step 202 includes blood glucose levels measured from capillary blood sample. In another embodiment, blood glucose may be estimated using plasma glucose samples, the means of measuring plasma glucose can be one of, but not limited to finger stick, saliva, tears and sweat. Blood glucose estimation from plasma, saliva, tears of sweat glucose measurements may be calculated using a mathematical model, the mathematical model may include but is not limited to Monte Carlo Tree Search, Neural Network Optimization or any other Artificial Intelligence. In some examples, blood glucose may also be estimated using a continuous glucose monitoring (CGM) system. The mathematical model may include one or more personally adaptive factors that may change over time.

The step 202 further includes creating historical data of the individual for the predefined period, wherein historical data of the individual is further analyzed for the predefined period to estimate a future trend of prediabetes or diabetes. The future trend of diabetes may be indicative of a possible trigger or onset of prediabetes or diabetes and associated risks. The historical data of the individual created in lieu of the personal data captured by data capturing module 102, may be further employed in creating patterns of the individual.

Accordingly, the personal data utilized at step 202, includes but is not limited to body mass index (BMI), waist circumflex (WC), sex, age, physical activity details, eating habit details, lifestyle details, genome related information and any other existing diseases' details. In some examples the personal data may be consumed by the mathematical model as personal factors in determining module 106.

Thereafter, at step 204, an insulin production trend and relative insulin resistivity is derived by deriving module 104 over the predefined period.

At step 206, a status of the onset and progress of prediabetes or diabetes in the individual is determined by determining module 106 wherein the status of prediabetes or diabetes in the individual is based on at least one of the relative insulin resistivity trend, insulin production trend for the individual, BMI, WC and personal data of the individual or combinations thereof.

Once the status of prediabetes or associated risk of diabetes has been determined in the individual, suggestions to the individual pertaining to at least one of medication prescriptions, medication dosages, eating habits and lifestyle changes for controlling progress of prediabetes or diabetes is provided. The suggestions regarding eating habits and lifestyle changes may be given on a real-time basis during the predefined period. For instance, in some examples, certain scenarios can be anticipated in terms of trend categories. When the insulin level is increasing and blood glucose level is also increasing, the individual is suggested and advised on changes for reducing insulin resistivity. When the insulin level is moderate and blood glucose level is increasing, the suggestions include prescribing medications to support the individual's own insulin production. In a similar fashion, when the insulin levels are increasing and have reached very high levels and the blood glucose levels are simultaneously increasing, the suggestions include extensive lifestyle changes to reduce insulin resistivity apart from advising add-on insulin to be administered to support body's insulin production.

In some examples, the suggestions can further include the intervention of a health care professional for better management of prediabetes and enable further diagnosis and prognosis to delay the onset of diabetes and its associated risks.

In a typical example in accordance with the present invention, monitoring the onset and progression of prediabetes is based on the identification of four trends established between fasting blood glucose (fBG) measurements combined with insulin (INS) level measurement of an individual. The four trends in view of normal/threshold levels of glucose (fBGₜₕ) and insulin (INSₜₕ) measurement and associated actions and suggestions provided to the individual are as follows:

1) fBG<fBGₜₕ AND INS<INSₜₕ=>no further actions at the moment;
2) fBG>fBGₜₕ AND INS>INSₜₕ=>T2D to be confirmed using normal medical routines;
3) fBG>fBGₜₕ AND INS<INSₜₕ=>T1D to be confirmed using normal medical routines; and
4) fBG<fBGₜₕ AND INS>INSₜₕ=>potential T2D prediabetes in the early stage.

In case of the fourth trend wherein the fasting blood glucose level is on normal level while insulin level is increased, the individual is a potential candidate for the onset of prediabetes. In such a scenario an individual may be suggested to repeat fasting blood glucose and insulin level measurements in the ensuing mornings and another measurement after a week. The other suggestions given to the individual include increasing physical activity level; maintain an appropriate diet, etc. The measured fasting blood glucose (fBG) and insulin level (INS) measurements may be used to calculate and follow a relative insulin resistivity trend (IR(t)) of the body via the ratio INS(t)/fBG(t), . . . , INS(t+N)/fBG(t+N) over a measurement period of N days, wherein (t) is a reference measurement's time stamp and (t+N) equals to time stamps over N days. In a scenario where IR(t+N)<IR(t), it may be established that the suggestions provided to the individual have proved to be beneficial. The individual is further suggested to continue following the suggestions and actions and take measurements one to two times per week until both fBG and INS levels are under fBGₜₕ and INSₜₕ, respectively. In a scenario where IR(t+N)≈IR(t) the individual may be suggested to take measurements once every six months to enable monitoring of any probable change in the scenario. If there further exists a scenario where IR(t) sometimes later is bigger than IR(t=1), the measurement pattern is repeated and a new intervention may be planned by a healthcare team according to the individual's needs.

Accordingly, in another embodiment an increasing trend like IR(t+M1), IR(t+M2), . . . , IR(t+Mn), wherein M is a longer duration of time period like a week, month, etc. may be be captured and stored in a reference database further comprising trends of other individual participants. The increasing trend of the individual may be compared with personal data of the other individual participants, wherein the other personal data includes but is not limited to blood glucose values, insulin level, body mass index (BMI), waist circumference, age, etc. based on any statistical model to determine any possible future development in the individual's condition in different life style scenarios. The possible future development as determined for the individual may be presented to the individual as a feedback thereby enabling the individual to analyze the appropriate life style scenario to follow.

In an exemplary embodiment, salivary insulin measured by non-invasive means and blood glucose measurements are captured by data capturing module 102 at predetermined time intervals for a predefined period. In another embodiment, the glucose measurements are measured from a salivary sample. In some examples, an individual having a personal account on a cloud based server, storing module 108 of system 100 is configured to store the measurements of salivary insulin, blood glucose or salivary glucose data, body mass index (BMI), waist circumflex and other personal data of the individual. The predefined period for data capturing is based on the threshold set as per baseline measurements of insulin and glucose related data. At deriving module 104, the trends namely for example, fasting salivary glucose or fasting blood glucose before breakfast (FSG/FBG), fasting salivary insulin before breakfast (FSI), and relative insulin resistivity (FSI/FBG) is derived in view of body mass index (BMI), waist circumflex (WC) and other personal data, for the ensuing weeks with regards to the baseline measurements. At determining module 106, the status, onset and progress of prediabetes or diabetes can be estimated by following any one of the given trends or combinations thereof and can be displayed on display module 110. Display module 110 can be communicatively couple to a personal device accessed by the individual, or can be an application in the device itself. Based on the trends and changes in the trend, appropriate suggestions may be provided to the individual. The suggestions may include changes in eating habits, lifestyle changes, prescription of medication, add-on insulin administration, intervention of a healthcare professional, etc.

Furthermore, historical data created by the data capturing module may estimate future trends of the individual to monitor the effectiveness of suggestions made to the individual, wherein the effectiveness is a relative measure of the progress of delaying the onset of diabetes in a prediabetic individual. Accordingly, further changes or modifications in the suggestions can be initiated by analysing the historical data stored in storing module 108.

In an embodiment of the present invention may relate to a computer program product with a non-transitory computer readable storage medium having computer code thereon for performing various computer-implemented operations of the method and/or system disclosed herein. The media and computer code may be those specially designed and constructed for the purposes of the method and/or system disclosed herein, or they may be of the kind well known and available to those having skill in the computer software arts. Examples of computer-readable media include, but are not limited to, magnetic media, optical media, magneto-optical media and hardware devices that are specially configured to store and execute program code. Examples of computer code include machine code, such as produced by a compiler, and files containing higher-level code that are executed by a computer using an interpreter. For example, an embodiment of the present invention may be implemented using JAVA®, C++, or other object-oriented programming language and development tools. Aspects of the present invention may also be implemented using Hypertext Transport Protocol (HTTP), Procedural Scripting Languages and the like.

Those skilled in the art will realize that the above-recognized advantages and other advantages described herein are merely exemplary and are not meant to be a complete rendering of all of the advantages of the various embodiments of the present invention. Additionally, embodiments need not achieve all these, or another advantage, and should not be limited there to.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as critical, required, or essential features, of the present invention.

What is claimed is:

1. A computer implemented method for monitoring an onset and progress of prediabetes in an individual, the computer implemented method comprising:
capturing, periodically, by one or more processors, an insulin level of the individual and a blood glucose level of the individual over a predetermined time interval for a predefined period;
deriving, by one or more processors, at least one of a insulin production trend and relative insulin resistivity trends over the predefined period, wherein the relative insulin resistivity is a ratio of an insulin level and a blood glucose level, wherein the trends are indicative of trend categories comprising an increasing trend, a steady trend and a decreasing trend; and
determining, by one or more processors, a status of prediabetes in the individual based on at least one of the insulin production trend, relative insulin resistivity trend, and personal data of the individual.

2. The computer implemented method of claim 1, wherein, the step of capturing further comprises non-invasive means of measuring insulin level by measuring C-peptide levels, wherein, insulin level and C-peptide level is measured from at least one of a blood sample, a saliva sample, a tears sample and a sweat sample.

3. The computer implemented method of claim 1, wherein, the relative insulin resistivity is a ratio of a salivary insulin level and a blood glucose level.

4. The computer implemented method of claim 1, wherein the step of capturing further comprises creating historical data of the individual for the predefined period.

5. The computer implemented method of claim 3, wherein the method for monitoring the progress of prediabetes further comprises analyzing historical data of the individual for the predefined period to estimate a future trend of diabetes.

6. The computer implemented method of claim 1, wherein personal data of an individual comprises at least one of body mass index (BMI), waist circumflex (WC), sex, age, physical activity details, eating habit details, lifestyle details, genome related information and any other existing diseases details.

7. The computer implemented method of claim 1 further comprising providing suggestions to the individual pertaining to at least one of medication prescriptions, medication dosages, eating habits and lifestyle changes for controlling progress of prediabetes.

8. The computer implemented method of claim 6 further comprising establishing effectiveness of the suggestions by monitoring variations in at least one of the insulin production trend and the relative insulin resistivity trends of the individual in response to the individual following at least one of the medication prescriptions, medication dosages, eating habits and the lifestyle changes.

9. The computer implemented method of claim 7 further comprising modifying suggestions based on the effectiveness, wherein suggestions are modified for at least one of reducing insulin resistivity, prescribing medication to regulate insulin production, prescribing add-on insulin and combinations thereof.

10. A system for monitoring an onset and progress of prediabetes in an individual, the system comprising:
a data capturing module periodically capturing insulin level of the individual, a blood glucose level of the individual, body mass index (BMI), waist circumflex (WC) and personal data of the individual at predetermined time intervals over a predefined period;
a deriving module deriving at least one of insulin production trend and relative insulin resistivity trends over the predefined period, wherein a relative insulin resistivity is a ratio of an insulin level and a blood glucose level; and
a determining module determining a status of prediabetes in the individual based on at least one of the relative insulin resistivity trend, insulin production trend for the individual, BMI, WC and personal data of the individual.

11. The system of claim 10 further comprising a display module displaying at least one of the insulin production trend and the relative insulin resistivity, wherein the trends are indicative of trend categories comprising an increasing trend, a steady trend and a decreasing trend.

12. The system of claim 10 further comprising a storing module for storing the insulin level of the individual, the blood glucose level of the individual, BMI, WC and personal data of the individual.

13. The system of claim 10, wherein personal data of the individual further comprises at least one of sex, age, physical activity details, eating habit details, lifestyle details, genome related information and any other existing diseases details.

14. The system of claim 12 wherein the storing module may be located in a computing device of the individual, wherein a computing device is at least one of a smartphone, a tablet, a laptop, a desktop, a wearable computer, a smartwatch, or a combination thereof.

15. The system of claim 12 wherein the storing module is located on a cloud based server.
